(19) United States
(12) Patent Application Publication
(10) Pub. No.: US 2020/0187981 A1
(43) Pub. Date: Jun. 18, 2020

(54) MULTI-TRANSDUCER ULTRASONIC TOOL-GUIDANCE

(71) Applicant: ULTRASEE CORPORATION, Palo Alto, CA (US)

(72) Inventors: Mu TIAN, San Jose, CA (US); Todd ZHOU, San Jose, CA (US); Yong TIAN, San Jose, CA (US)

(73) Assignee: ULTRASEE CORPORATION, Palo Alto, CA (US)

(21) Appl. No.: 16/608,486

(22) PCT Filed: Apr. 26, 2018
(86) PCT No.: PCT/US2018/029526

§ 371 Date: Oct. 25, 2019

Related U.S. Application Data
(60) Provisional application No. 62/589,774, filed on Nov. 22, 2017.
(60) Provisional application No. 62/490,440, filed on Apr. 26, 2017.

(51) Int. Cl. A61B 17/34; A61B 8/00; A61B 8/08; A61B 90/00

(52) U.S. Cl. CPC A61B 17/3403; A61B 8/4477; A61B 8/463; A61B 2090/372; A61B 8/0841; A61B 90/37; A61B 2017/3413; A61B 8/4494

(57) ABSTRACT

In an ultrasonic tool-guidance system, an imaging module in a housing includes modules for image and beam processing and beamforming. A transmitter and a receiver connect via a switch to a multi-transducer probe. The housing includes a display for providing real-time ultrasound images. The probe includes a tool-guiding channel through which a tool can be inserted into the body of a patient. The transducers are arranged at an angle laterally around the tool-guiding channel. The angled transducers provide overlapping beams that result in a better quality enhanced image of the center area. The probe is rotatably attached to the housing to allow for easy viewing of the ultrasound images during a procedure. The tool-guiding channel is maintained at a fixed angle with respect to the transducers and the probe to assure that the tool remains in the illuminated area throughout the procedure.

### CROSS-REFERENCE TO RELATED APPLICATIONS

This application claims priority to U.S. Provisional Patent Application No. 62/490,440 titled, “Dual Angled Transducer Beamforming,” filed on Apr. 26, 2017 and to U.S. Provisional Application No. 62/589,774 titled, “Precise Needle Guidance Device,” filed on Nov. 22, 2017, both of which are incorporated by herein by reference in their entirety.

### BACKGROUND OF THE INVENTION

This disclosure generally relates to ultrasound systems, and more specifically to ultrasonic guidance systems for use in medical procedures requiring the guidance of tools through body tissues.

Ultrasound systems have become widely-used diagnostic tools for various medical applications. Many ultrasound systems, compared to some other diagnostic tools or systems, are non-invasive and non-destructive. An ultrasound system generally includes a probe for approaching or placing directly on and moving over a subject, such as a patient. The ultrasound system may provide visualization of the subject's internal structures, such as tissues, vessels, and/or organs. The ultrasound system works by electrically-exciting transducer elements inside the probe to generate ultrasound signals, which travel into the body, and by receiving the echo signals reflected from tissues, vessels, and/or organs. The reflected echo signals are then processed to produce a visualization of the subject's internal structures.

One of the applications of ultrasound systems is to provide visual guidance to medical practitioners during procedures involving the insertion of tools into a patient's body tissues. For example, biopsies, minor surgical procedures, placement of intra-venous tubes for delivery of drugs, insulin, etc., or for injecting sub-cutaneous tissues with drugs or other treatments. In these applications, typically, the medical practitioner sees inserted needles appears at some location determined by the inserting angle, the distance between the inserting point and the probe, and the inserting depth. In order to practice interventional medical procedure, such as needle injection or biopsy, the operator needs to find the target, mark it, pre-compute the inserting angle, align with the line of direction or adjusting the needle guides' direction at a certain angle, such that the needle won't miss the target. The procedure is complex, the needle is hard to detect and display. There are a few constantly changing variables that make precise operation extremely difficult. Most solutions, like magnetic location needle display, puncture rack guidance, and the like are designed for improving these procedures, but unfortunately are not very successful.

For example, with current uses, it remains difficult to track where the needle tip is because the handheld probes shift with respect to the tool-handling hand. In addition, the inaccuracy of the probe location with respect to the tool due to for example, the ability to tilt and rotate the hand-held probe while handling also contribute to the difficulty in precisely locating the inserted tool. This typically can cause longer operating times and patient pain.

What is needed is an ultrasound-based tool-guiding system that addresses the deficiencies of the prior art to guidance so that the user can accurately see in real time the tool as it gradually penetrates and precisely reaches the target tissues.

### BRIEF SUMMARY

According to various embodiments of the present invention, an ultrasound-based tool-guidance system and method are provided.

In one embodiment, an apparatus is provided for real-time multi-beam ultrasound imaging used for guidance of a tool during a procedure. The apparatus includes a transducer container including a plurality of transducers, and a tool-guiding channel for receiving an insertion tool for use during the procedure. A housing of the apparatus includes ultrasound beam processing and image processing circuits and a display. The transducer container is rotatably coupled to the housing.

According to one embodiment, the plurality of transducers are spaced laterally around the tool-guiding channel and are angled inboard in relation to the bottom surface of the transducer container. The bottom surface of the transducer container is adapted to allow transmission of ultrasound signals from the plurality of transducers and is also arranged at a fixed angle with respect to a longitudinal axis of the tool-guiding channel.

According to another embodiment, the ultrasound signals from the plurality of transducers may be arranged to form an ultrasonic beam processed and to form a single ultrasound image for displaying on the display. In one embodiment, the angle formed between each of the plurality of transducers and the bottom surface of the transducer container is within the range of five to fifty-five degrees. Further, according to another aspect of one embodiment, this angle allows the detection of liquid flow within a body of a patient.

In another embodiment, the bottom surface of the transducer container is substantially perpendicular to the longitudinal axis of the tool-guiding channel. In one embodiment, the plurality of transducers are arranged to detect a position of the insertion tool that is inserted through the tool-guiding channel through skin of a patient. In alternative embodiments, the insertion tool may be a needle or a cutting tool used for medical procedures.

In one embodiment, the apparatus for providing real-time multi-beam ultrasound imaging for guidance of a tool may also include a body-attachment mechanism coupled to the housing for attaching the apparatus to a body of a patient during the procedure. The body-attachment mechanism be, for example, one of a belt or a tape.

In one embodiment, the apparatus for providing real-time multi-beam ultrasound imaging for guidance of a tool may also include a lockable attachment mechanism configured to allow locking of the rotatably attached transceiver container at a fixed position. In one embodiment, the tool-guiding channel includes an opening for receiving the insertion tool that has a diameter between 1 mm and 10 mm.

The apparatus may be configured to be water proof and/or to be resistant to shocks or vibrations.

According to another embodiment, the apparatus for providing real-time multi-beam ultrasound imaging for guidance of a tool includes a gel layer extending at least partially over the outside surface of the transmitter container. In one embodiment, the transducer container is a disposable attachment. In one embodiment, the gel layer provides a cushion between the apparatus and a patient during the procedure. The gel layer may be made of a medical grade silicone and may include a cylindrical component that extends along an outside surface of the tool-guiding channel. In different embodiments, the gel layer may also include one or more disposable components.

### BRIEF DESCRIPTION OF THE SEVERAL VIEWS OF THE DRAWINGS

FIG. 1 is a block diagram of an ultrasonic tool guidance system in accordance with various embodiments.

FIG. 2 is a diagram of an ultrasound tool-guidance system according to one embodiment.

FIG. 3 is a cross-sectional view of a three-dimensional illustration of a coupling gel layer according to one embodiment.

FIG. 4 a schematic diagram of an ultrasonic tool-guiding system according to one embodiment.

FIG. 5 illustrates an exemplary ultrasound-based needle-guidance device according to one embodiment.

FIG. 6 illustrates an exemplary ultrasound-based needle-guidance device according to another embodiment.

FIG. 7 is a block diagram of an image module in an ultrasound-based tool-guidance system according to one embodiment.

FIG. 8A illustrates a real-time ultrasound image of a tool insertion procedure with a tool-guidance device according to one embodiment.

FIG. 8B illustrates a real-time ultrasound image of a tool insertion procedure with a tool-guidance device according to one embodiment.

FIG. 8C illustrates a real-time ultrasound image of a tool insertion procedure with a tool-guidance device according to one embodiment.

The figures depict various example embodiments of the present disclosure for purposes of illustration only. One of ordinary skill in the art will readily recognize form the following discussion that other example embodiments based on alternative structures and methods may be implemented without departing from the principles of this disclosure and which are encompassed within the scope of this disclosure.

### DETAILED DESCRIPTION

A detailed description of one or more example embodiments of a system and method is provided below along with accompanying figures. While this system and method is described in conjunction with such embodiment(s), it should be understood that the system and method is not limited to any one embodiment. On the contrary, the scope of the system and method is limited by the claims and the system and method encompasses numerous alternatives, modifications, and equivalents. For the purpose of example, numerous specific details are set forth in the following description in order to provide a thorough understanding of the present system and method. These details are provided for the purpose of example, and the system and method may be practiced according to the claims without some or all of these specific details.

For the purpose of clarity, technical material that is known in the technical fields related to the system and method has not been described in detail so that the present system and method is not unnecessarily obscured.

A system is described for performing ultrasound imaging for guidance of medical instruments, such as for example a needle. Various embodiments may be implemented in discrete hardware components or, alternatively, in programmed processing units such as digital signal processors using software which is compiled, linked and then loaded from disk-based storage for execution during run-time. Various programs including the methods employed in these embodiments may also reside in firmware or other similar non-volatile storage means.

It should also be appreciated that the present system and method may be implemented in numerous ways, including as a process, an apparatus, a device, or a computer-readable medium such as a non-transitory computer-readable storage medium containing computer-readable instructions or computer program code, or as a computer program product, comprising a non-transitory computer-usable medium having a computer-readable program code embodied therein. In the context of this disclosure, a computer-usable medium or computer-readable medium may be any non-transitory medium that can contain or store the program for use by or in connection with the instruction execution system, apparatus or device. For example, the computer-readable storage medium or computer-usable medium may be, but is not limited to, a random access memory (RAM), read-only memory (ROM), or a persistent store, such as a mass storage device, hard drives, CDROM, DVDROM, tape, erasable programmable read-only memory (EPROM or flash memory), or any magnetic, electromagnetic, infrared, optical, or electrical means or system, apparatus or device for storing information. Alternatively or additionally, the computer-readable storage medium or computer-usable medium may be any combination of these devices. Applications, software programs or computer-readable instructions may be referred to as components or modules. Applications may be hardwired or hard coded in hardware or take the form of software executing on a general-purpose computer or be hardwired or hard coded in hardware such that when the software is loaded into and/or executed by the computer, the computer becomes an apparatus for practicing the system and method. Applications may also be downloaded, in whole or in part, through the use of a software development kit or toolkit that enables the creation and implementation of the present system and method. In this specification, these implementations, or any other form that the system and method may take, may be referred to as techniques. In general, the order of the steps of disclosed processes may be altered within the scope of the system and method.

Various embodiments of ultrasound apparatuses and methods are described. It is to be understood that the invention is not limited to the particular embodiments described as such which may, of course, vary. An aspect described in conjunction with a particular embodiment is not necessarily limited to that embodiment and may be practiced in any other embodiments. For instance, while various embodiments are described in connection with ultrasound machines, it will be appreciated that the invention can also be practiced in other imaging apparatuses and modalities. It is also to be understood that the terminology used herein is for the purpose of describing particular embodiments only, and is not intended to be limiting since the scope of the invention will be defined by the appended claims, along with the full scope of equivalents to which such claims are entitled. In addition, various embodiments are described with reference to figures. It should be noted that the figures are intended to facilitate the description of specific embodiments and they are not intended as an exhaustive description or as a limitation on the scope of the invention.

Various relative terms such as “upper,” “above,” “top,” “over,” “on,” “below,” “under,” “bottom,” “higher,” “lower” or similar terms may be used herein for convenience in describing relative positions, directions, or spatial relationships in conjunction with the drawings. The use of the relative terms should not be construed as to imply a necessary positioning, orientation, or direction of the structures or portions thereof in manufacturing or use, and to limit the scope of the invention. As used in the description and appended claims, the singular forms of “a,” “an,” and “the” include plural references unless the context clearly dictates otherwise.

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

Although various embodiments are described herein with reference to ultrasound imaging of various anatomic structures, it will be understood that many of the methods and devices shown and described herein may also be used in other applications, such as imaging and evaluating non-anatomic structures, animals, and objects. For example, the ultrasound probes, systems and methods described herein may be used in non-destructive testing or evaluation of various mechanical objects, structural objects or materials, such as welds, pipes, beams, plates, pressure vessels, layered structures, etc. Furthermore, the various embodiments of systems and methods for assessing movement or velocity of an imaged object or substance may also be applied to non-medical scenarios such as guidance of tools for making reparations to a pipe, pressure vessel or other conduit or container. Therefore, references herein to medical or anatomic imaging targets such as blood, blood vessels, heart or other organs are provided merely as non-limiting examples of the nearly infinite variety of targets that may be imaged or evaluated using the various apparatus and techniques described herein.

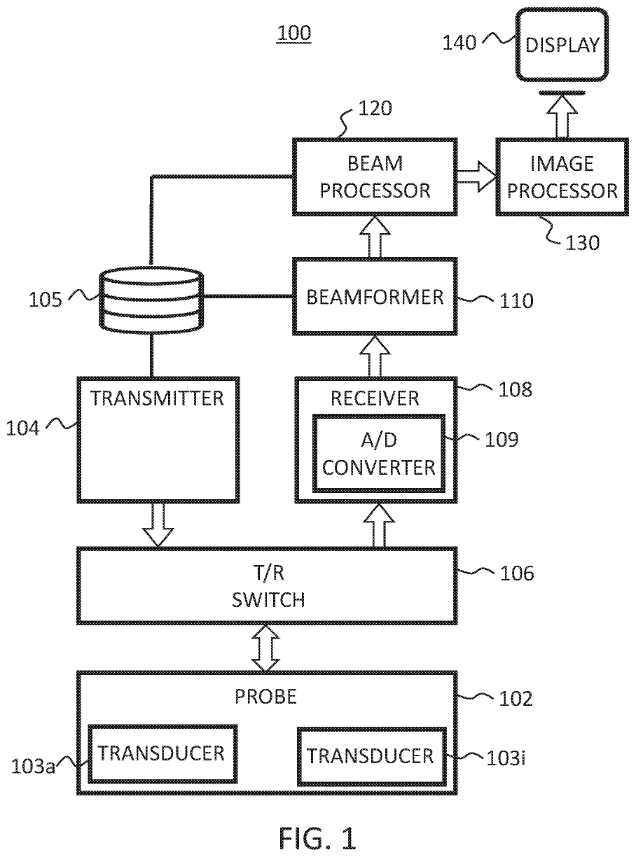

Referring now to FIG. 1, a block diagram of an ultrasonic tool guidance system 100 is disclosed in accordance with various embodiments. The exemplary system 100 may include an ultrasound probe 102, a transmitter/receiver switch 106 operatively coupled to the probe 102, a transmitter 104 operatively coupled to the transmitter/receiver switch 106, a receiver 108 operatively coupled to the transmitter/receiver switch 106, a beamformer 110 operatively coupled to the receiver 108, a receiving beam processor 120 operatively coupled to the beamformer 110, an image processor 130 operatively coupled to the receiving beam processor 120, and a display unit 140 operatively coupled to the image processor 130. The ultrasound probe 102 may be a probe used in contact with a subject for ultrasound imaging. The ultrasound probe 102 may include a plurality of ultrasound transducer elements 103a . . . 103i. Suitable configurations of probe 102 with the transducer elements 103a . . . 103i inside may include, but not limited to, linear, curved (e.g., convex), among others.

The exemplary ultrasound imaging system 100 may also include a memory 105. The memory 105 may include volatile or non-volatile digital memory storage device. In embodiments, the memory 105 may also comprise communication electronics for transmitting data to an external device over a wired or wireless connection or network. In other embodiments, the memory device 105 may include a combination of volatile memory, non-volatile memory and communication electronics. Though in FIG. 1 the memory device 105 is shown as a single device, the memory device 105 may be a plurality of devices available for access by and operatively coupled to the transmitter 104, the beamformer 110, and the receiving beam processor 120, among others. Though not shown in FIG. 1, in embodiments, the memory 105 may be operatively coupled to the receiver 108 to store raw data for later processing.

In embodiments, the image processor 130 may include any digital signal processing and/or computing components configured to perform the specified processes. For example, in various embodiments the functionality of the image processor 130 may be performed by software or firmware executed by a processor that may be shared for other computing functions. In one embodiment, the processor that runs the image processor software is a GPU. In another embodiment, the image processor firmware runs on a FPGA architecture. The image processor 130 may include any video and/or audio processing hardware, firmware and software components that may be configured to assemble image frames into a video stream for display and/or storage.

As used herein the term “ultrasound transducer element” and “transducer element” may carry their ordinary meanings as understood by those skilled in the art of ultrasound imaging technologies, and may refer, without limitation, to any single component capable of converting an electrical signal into an ultrasonic signal and/or vice versa. For example, in embodiments, an ultrasound transducer element may comprise a piezoelectric device. Other types of ultrasound transducer elements may also be used in place of a piezoelectric device.

As used herein, the term “transmit element” may refer without limitation to one or a few ultrasound transducer elements, which at least momentarily perform a transmit function in which an electrical signal is converted into ultrasound wave. Similarly, the term “receive element” may refer without limitation to one or a plurality of ultrasound transducer elements, which at least momentarily performs a receive function in which an ultrasound wave impinging on the one or the plurality of elements is converted into an electrical signal. Transmission of ultrasound into a medium may also be referred to herein as “illuminating.” An object or structure which reflects ultrasound waves may be referred to as a “reflector” or a “scatterer.” The reflector may be identified as one or more points. A point may be referred to as a position or a location within the region of interest. And the point may be presented as one or more pixels on the display 140 of the ultrasound image.

In embodiments, echo data may be received, beamformed, processed and displayed in substantially real-time, while simultaneously being stored in the memory device 105. In embodiments, processing and/or beamforming for real-time display may include retrieving echo data resulting from multiple transmit events from the memory device 105 (which may operate in a buffer mode), and beamforming or processing may be performed simultaneously on echo data received from a plurality of signals transmitted at different times. In embodiments, echo data may be stored in a long-term memory storage device, and may be beamformed and processed for display at a later time, and/or used by different computing hardware than the system 100.

An ultrasound imaging process may begin with a selection of one or more transducer elements 103 as a transmit (TX) element. Though not shown in FIG. 1, the transmit element may be selected by a transmit control unit. The transmit control unit may be part of the transmitter 104 and residing on the transmitter 104 in embodiments. In various embodiments, the transmit control unit may be a separate unit residing independently or on other components of the exemplary imaging system 100. Upon selection by the transmit control unit, the transmit control unit may store information about the transmit event and the transducer element(s) used during each transmit event in the memory 105.

As used herein, a transmit event may include using one transducer element to repeatedly generate a plurality of waves that transmit ultrasound energy into the region of interest. A round of transmit may include multiple transmit events sequentially emitted incrementally across the width of the probe face, thus interrogating an entire image frame. In a round of transmission, information may be recorded as transmit data. Combining with receiving beam data, the data from one round of transmission may be used to produce one complete image frame. The transmit information, such as attributes of the transducer element including the spacing, as well as a frequency, magnitude, pulse length, among others may be recorded as transmit data by the transmit control unit. Transmit data is collectively referred herein to as “TX data”.

Once a transmit element is selected, a sequence of high voltage pulses may be generated by the transmitter 104 operatively coupled to the transmitter/receiver switch 106. As used herein the transmitter may be referred to as pulser. The high voltage pulses generated by the transmitter 104 may go through the transmitter/receiver switch 106 to the transducer elements 103a . . . i inside the probe 102 and may be converted to ultrasound wave by the selected transmit element comprising one or more transducer elements 103a . . . i. Though transmitting ultrasound waves requires high voltage pulses, receiving echoes of the ultrasound waves may need low voltage signals. The transmitter/receiver switch 106, operatively coupled to the probe 102, may prevent the high voltage pulses from damaging the receive electronics in the receiver 108. Thus, by having the transmitter/receiver switch 106 operatively coupled to the probe 102, the transducer elements 103a . . . i may function as both transmit elements and receive elements. When there is a high voltage pulse, a transducer element may be used as a transmit element to generate ultrasound. When echoes propagate back to the probe 102, the same transducer element may function as a receive element to collect echoes as low voltage signals and the collected low voltage signals may then go through the transmitter/receiver switch 106 before being converted to digital numbers by the receiver 108.

Referring to FIG. 1, as the transmitted ultrasound waves illuminate the region of interest, they may migrate through materials with different densities. With each change in density, the ultrasound waves may have a slight change in direction and produce a reflected ultrasound wave as an echo. Some of the echoes may propagate back to the transducer elements 103a-i and may be captured as low voltage signals by the transducer elements 103a-i. The transducer elements 103a-i may pass the low voltage signals to the receiver 108.

The receiver 108 may include an analog/digital (A/D) converter 109 residing on the receiver 108 or otherwise operatively coupled with the receiver 108, for example as a separate chip within a package or module (e.g., multi-chip module) or in a different package. Though not shown in FIG. 1, in addition to the analog/digital converter 109, the receiver 108 may include receiving circuits, low-voltage differential signaling (LVDS) bridges among others according to embodiments. Upon receiving the electronic signal, the analog/digital converter 109 may convert the electronic signal to digital numbers. In embodiments, the conversion may be performed by firmware running on a field-programmable gate array (FPGA). After generating the digital numbers, the receiver 108 may route the output to the beamformer 110. Though not shown in FIG. 1, in embodiments, the receiver 108 may store the output to the memory 105 and the data may be obtained by or provided from memory 105 to the beamformer 110.

In one embodiment, the beamformer 110 may include additional components to scale the receiver input and perform additional signal processing to form the output beam. For example, while not shown in FIG. 1, the beamformer 110 may include a channel delay control module, a channel first-in-first-out (FIFO) memory, and a summation module. The channel delay control module may scale the output from the receiver 108 by introducing delays to the digital numbers. The output from the channel delay control module may be stored in the channel FIFO memory, which may be separate from or a part of memory 105. A summation module may perform the summing of the data stored in the channel FIFO memory to form a set of receiving beams. In embodiments, the beamformer 110 may be implemented in an application-specific integrated circuit (ASIC), a field-programmable gate array (FPGA), digital signal processor (DSP), or a combination of these components.

The data corresponding to the set of receiving beams from the beamformer 110 may be stored in the memory 105. The stored receiving beam data may be retrieved immediately or at a later time and sent to the receiving beam processor 120. The receiving beam data is collectively referred herein to as “RX data”. The RX data may include a receiving beam index associated with each receiving beam indicating the location of the receiving beam in the set of receiving beams. In embodiments, the RX data may be stored then modified during and/or after beamforming and generated as a data set including both the TX data and RX data. The data set may be collectively referred herein to as “beam data”. In various embodiments, the TX and RX data may be stored separately and cross reference each other.

In embodiments, the memory 105 may comprise a temporary buffer (volatile or non-volatile) to store intermediate calculation result for faster access and reproduction of images in the display 140. For example, data for color Doppler imaging or B-mode imaging may be stored in the temporary buffer for faster access. In embodiments, if the processing hardware is sufficient to hold the data and use the data for the imaging processing, the step of storing the position data may be omitted. For example, to generate a Doppler image, upon receiving the beam data, the beam processor 120 may process the beam data and send the processed data to the image processor 130. To generate B-mode image, upon receiving the beam data, the beam processor 120 may group the beam data and sum the data before send to the image processor 130 to form B-mode image data. The processed beam data from beam processor 120 may be stored in the memory 105 and/or sent to the image processor 130 and displayed at the display unit 140.

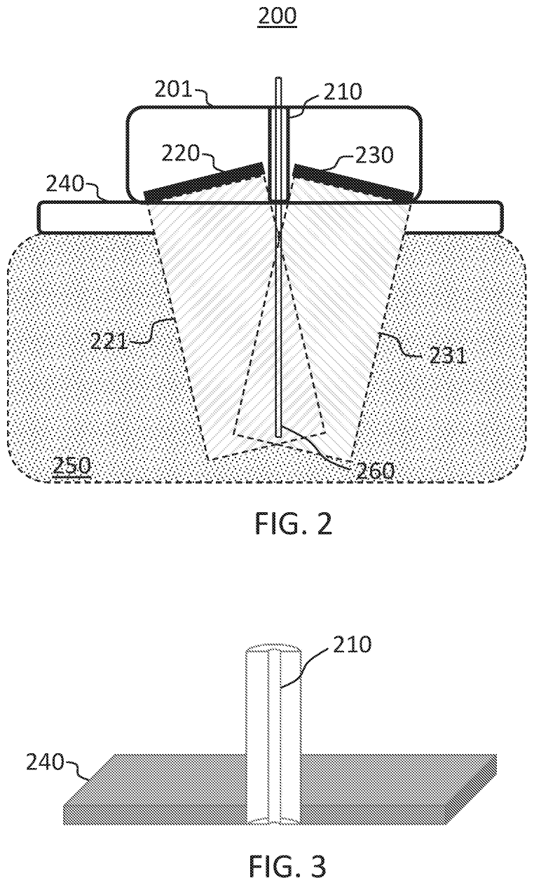

Referring now to FIG. 2, a diagram of an ultrasound tool-guidance system 200 according to one embodiment is shown. In one embodiment, a probe 201 includes a plurality of angled transducers. Suitable configurations of probe 201 with the transducer elements inside may include, but not limited to, linear, curved (e.g., convex), among others. For example, in one embodiment, the probe 201 includes a left transducer 220 and a right transducer 230, with the transmissive surfaces facing at an angle towards each other and around a tool guiding channel 210. In this configuration, each of the transducers, e.g., left and right transducers forms an angle with the bottom of the probe 201, which may be within the range of five to fifty-five degrees, depending on the embodiment. The tool-guiding channel may be formed of different widths and shapes to accommodate different tools. For example, for needle guidance embodiments, the center channel may be substantially cylindrical with a diameter that may range from approximately 1 mm to 10 mm. In some embodiments, the probe 201 or the tool-guidance channel 210 are detachable and replaceable from a housing (not show) to allow for different uses.

In different embodiments, the transducers 220 and 230 may be provided in any form of linear array, or any type of convex array, or any type of concave array, for example. The transducers are arranged laterally around a central tool-guiding channel 210. In one embodiment, the tool-guiding channel 210 is configured to receive a transdermal needle. In an alternative embodiment, the tool-guiding channel 210 is configured to receive a cutting tool. In yet another embodiment, the tool-guiding channel 210 is configured to receive a vein cannulation tube and needle for intra-venous insertion applications.

Referring back to FIG. 2, by using two or more angled transducers 220, 230 (etc.) arranged facing the tool-guiding channel 210, the center area of the image produced by the transducers is enhanced from a resolution point of view. The imaged areas 221 and 231 overlap around the center area, where the target tissues are likely to interact with the guided tool. The Image processor module (as for example shown in FIG. 1, item 130), applies image processing algorithms to form a single image from the tissue areas 221 and 231 illuminated by the different transducers 220 and 230. The resulting beam data provides additional samples for the area of overlap in the center, resulting in better resolution and an enhanced image. For example, in a needle-based biopsy procedure application, the resulting enhanced image makes it easy to locate the target with a needle at the center where the biopsy is done. The needle is free to insert through the guiding channel 210. Since the transducers are angled, it is easier to detect the needle progressing through the tissues as compared with a conventional flat transducer arrangement. Additionally, because the ultrasonic transducers are angled with respect to the surface of the device and patient's skin, the system 200 can be used to sense and measure blood flow within the patient's vessels as for example described in co-pending U.S. patent application Ser. No. 14/550,096 (filed on Nov. 21, 2014), incorporated herein by reference.

The ultrasound tool-guidance system 200 also includes a coupling gel layer 240, that may optionally surround the tool-guiding channel 210. For example, FIG. 3 shows a cross-sectional view of a three-dimensional illustration of a coupling gel layer 240 that extends up around the tool-guiding channel 210 according to one embodiment. In one embodiment, the coupling gel layer 240 is a one-time-use attachment that can be easily replaced with removable fasteners (not shown). In one embodiment, different attachments with different shapes may be used to provide guidance for different tools or for different uses. The coupling gel layer 240 may be made of plastic, silicone, or similar synthetic materials. For example, in one embodiment, the coupling gel layer 240 is made of medical grade silicone, such as a silicone rubber manufactured by NuSil™ or similar materials. In an exemplary use, the probe 201 is placed against the patient's body 250 via a gel barrier 240. For example, in one embodiment, gel barrier 240 is 5 mm thick. A transdermal needle 260 is inserted in the needle channel 210 through the top opening of the needle channel 110 while the left transducer 220 and the right transducer 230 provide imaging guidance for the needle 260 through a display. According to some embodiments, the gel layer 240 is soft enough to allow some degree of compression so it can form an angle with different body surfaces. In one embodiment, the gel layer 240 has a shape that matches the tool guidance channel 210 in the probe 201. For example, as shown in FIG. 3, in one embodiment, the cylinder shape of a silicone gel layer 240 extending upwards to cover the tool-guidance channel 210 functions as holder of the tool to help prevent movement, and also provides protection against contamination of the probe.

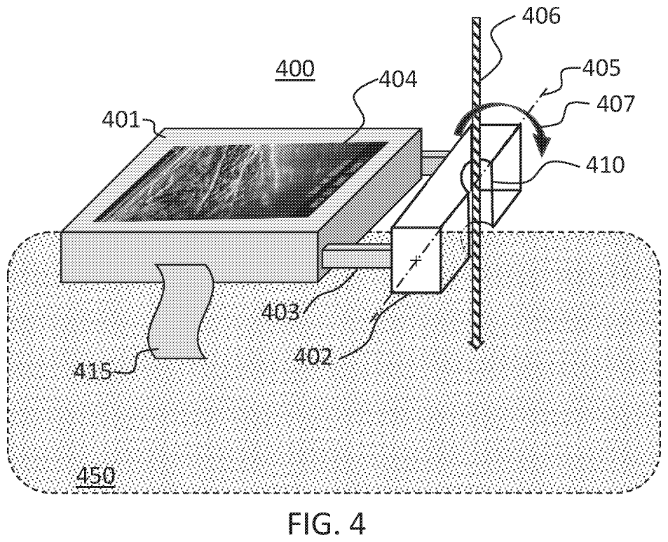

Now referring to FIG. 4, a schematic diagram of an ultrasonic tool-guiding system is shown according to one embodiment. The tool-guiding system 400 includes a display housing 401 to provide a real-time display or image 404 of the inner tissues 450 within a body part. The system 400 includes a probe 402 containing a plurality of transducers (not shown). The probe 402 includes a tool-guiding channel 410. The probe 402 is attached to the display housing 401 through a rotating attachment mechanism 403. According to this embodiment, as illustrated by arrow 407, the rotating attachment mechanism 403 allows the probe 402 to tilt or rotate along a longitudinal axis 405. For example, the probe 402 may rotate in either direction between 0 and 180 degrees. As illustrated in FIG. 4, the probe 402 is at 90 degrees of tilt. The rotating attachment mechanism 403 includes a locking feature, for example by clamping probe 402, that locks the probe at the desired tilt angle. By tilting the probe, tool-guiding channel 410 changes the direction at which a tool 406 may be inserted through the channel into the underlying tissues 450. However, the tool insertion direction remains perpendicular to (at approximately 90 degrees) to the probe surface facing the body part. This enables the system to maintain the tool in-plane with the image generated by the system 400 because the transducers (not shown) remain at a constant angle with respect to the tool 406. The inserted tool is guaranteed to be in one plane with the transducers, without extra installation of additional guidance track or shaft. As for example illustrated in FIG. 8A-8C, this allows for a clear image of the tool as it progresses through the tissues while being inserted as the tool remains at a fixed angle with respect to the transceivers during the insertion process.

For example, as illustrated in FIG. 2, the transducers 220 and 230 may be arranged at an angle with respect to the tool. But the rotation or tilt of the probe, as illustrated in FIG. 4, does not change that angle; the guiding-channel-transducers angle remains fixed.

According to one embodiment, the tool-guiding system 400 may include a body attachment mechanism 415, such as for example a belt or band. The display housing 401 can be attached to a body part, such as an arm, leg, or the like, allowing a hands-free operation and thereby enabling the operator to better handle tool 406 and other attachments or tools. For example, in one embodiment, too-guiding system 400 is used for intra-venous (“IV”) tube placement applications. By attaching the system 400 to a patient's arm, a medical professional can more easily maneuver a needle 406 and IV tube while looking at the display 404 to guide the placement of the IV needle in the vessel. In one embodiment, the housing 401 may be dimensioned to be attached to a human limb with attachment belt or band 415, such as an elastic, plastic, or leather band with an associated buckle, clip, or other loop closing means (e.g., Velcro™ or the like). For example, in one embodiment, housing 401 may be approximately 40 by 30 mm along its top surface holding the display 404 and of a depth of approximately 15 mm. Different sizes may be used in different embodiments to enable the system 400 to attach to a body part an enable hands-free operation as described above.

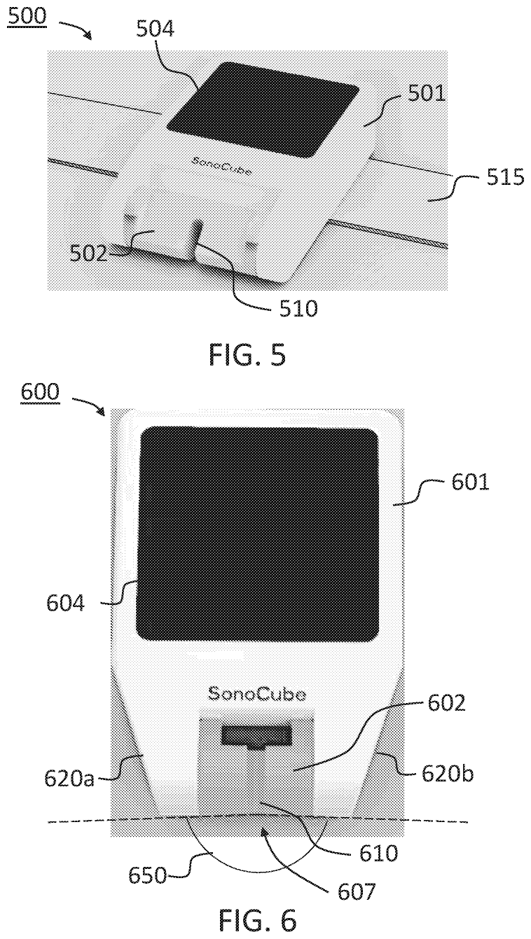

Now referring to FIG. 5, an exemplary ultrasound-based needle-guidance device 500 is shown according to one embodiment. In this embodiment, a probe 502 includes a needle-guiding channel 510 configured for placement of a needle. For example, needle-guidance system 500 may be used for intra-venous (“IV”) tube placement applications. In this embodiment, the narrow opening of the channel 510 is designed to retrieve the IV tube, or needle from the probe 502. The probe 502 is rotatably attached to housing 501. Housing 501 includes a display 504 and the ultrasound imaging components (not shown) discussed above with reference to FIG. 1. The display 504 is provided to display the ultrasound images generated by the system 500 in real time while allowing guidance of a needle inserted through the needle-guiding channel 510. The device 500 includes a band, tape, or belt 515 to attach the system to a patient's body part during use. The complete device 500 may be water proof and shock proof.

FIG. 6 provides another illustrative embodiment of a needle-guidance device 600. In this embodiment, the probe 602 is rotatably attached to the housing 601 at the end of tapered sides 620a and 620b In this embodiment, the housing 601 is dimensioned to be held in an operator's hand during use. In addition, the probe head 602 is specially designed for procedures involving human joints, such as a shoulder, ankle, or knee. In order to allow for in-plane injection of a needle into joint tissues, in this embodiment, the total length of the surface 607 of the probe 602 nearest to the patient is approximately 20 mm. The hole gap in the needle-guiding channel 610 is smaller, for example under 2 mm. The surface 607 intended for contact with the patient is not straight but rather forms an inner angle to allow maximum access to the joint, e.g., ankle, knee, shoulder, or the like, which typically present a round and hard surface against which the probe is placed. In this embodiment, the left and right transducers (not shown) form an angle 650 of approximately 145 to 175 degrees apart from each other. The probe 602 is attached to housing sides 620a and 620b allowing rotation around the axis perpendicular to the needle-guiding channel 610. For example, in one embodiment the probe 602 may be able to rotate between 0 and 90 degrees. In operation, this enables the operator to hold the housing 601 from a position perpendicular to the needle tool, when the probe is at 90 degrees, to essentially being in-line with the needle tool. For example, this enables a medical operator to hold a tool, such as a syringe, in one hand and the needle-guiding device 600 in the other, with the screen 604 facing the operator to see the needle going inside a patient's joint, e.g., a shoulder, and deliver an injected solution into the appropriate tissue as shown in the display 604. The complete device 600 may be water proof and shock proof.

In different embodiments different rotational degrees are possible. In different embodiments the probe 602 may be able to freely rotate, may be lockable, or may rotate at preset stops with increased friction between each rotating stop. For example, in one embodiment a needle-guidance system is used for a deep transdermal needle insertion, for example insertions deeper than 20 mm. Exemplary procedures for such use include anesthesia or biopsies. In this embodiment, the probe 602/502 may be used at angles between 45˜135 degrees, which would allow for ergonomic use of the device housing 501/601. In use, the user searches for the target guided by the ultrasound image provided in real-time via the display 504/604. Once the target tissue is found, the probe angle is locked. At this point, optionally, the device may be fixed to the patient's body by tape, belt, or the like, for “hands-free” operation. After the device is fixed, the probe angle can be fine-tuned or slightly re-adjusted for best insertion angle and then locked again for secure operation. The display screen 504/604 faces up towards the user leaving the user both hands to complete the procedure while looking at the ultrasound image for guidance to reach the desired tissue with the inserted needle. In an alternative use, for example, to guide an intra-venous needle and tube placement, the system 500/600 can provide guidance to reach deeper veins and assist with other difficult cases. In such uses, the probe 510/610 can tilt to over 135 to 175 degrees to provide easier access and to place the IV tube while maintain the display 504/604 facing the medical practitioner to provide needle guidance and optionally attaching the device to the patient for hands-free operation.

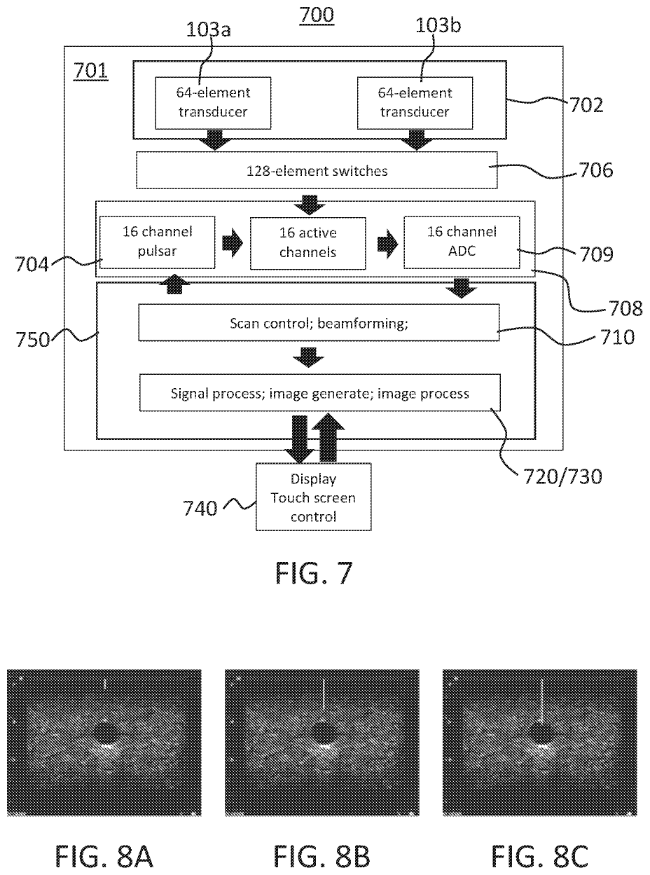

Now referring to FIG. 7, a block diagram of an image module in an ultrasound-based tool-guidance system according to one embodiment is provided. In this embodiment, the system 700 includes an image module 701 and a touch screen display 740. The image module 701 includes a probe 702 with two 64-element transducers 703a and 703b. The transducers are coupled to a 128-element switch 706. The image module 701 includes a 16 active channels transceiver 708. The transceiver 708 includes a transmitting module 704, for example, a 16-channel pulsar and a receiving module 709, including for example, a 16-channel A/D converter. The transceiver 708 is connected to a Field Programmable Gate Array (FPGA) 750 that includes logic to provide a beamformer 710 with scan control functionality. The FPGA 750 also includes logic to provide various digital signal processing modules, including a beam processor module 720 to process the beam signals, and an image processing module 730 to generate tissue image data and process the image data into a resulting ultrasound image. The ultrasound image is displayed on the touch-screen display 740. In alternative embodiments, the transducers can include any number elements, for example from 16 to 128 elements. Similarly, the number of active channels can vary, for example, between from 4 and 64 channels.

As those in the art will understand, a number of variations may be made in the disclosed embodiments, all without departing from the scope of the invention, which is defined solely by the appended claims. It should be noted that although the features and elements are described in particular combinations, each feature or element can be used alone without the other features and elements or in various combinations with or without other features and elements. The methods or flow charts provided may be implemented in a computer program, software, or firmware tangibly embodied in a computer-readable storage medium for execution by a general-purpose computer or a processor.

Suitable processors include, for example, a general-purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Arrays (FPGAs) circuits, any other type of integrated circuit (IC), and/or a state machine.

While several implementations have been provided in the present disclosure, it should be understood that the disclosed systems and methods may be implemented in many other specific forms without departing from the scope of the present disclosure. The present examples are to be considered as illustrative and not restrictive, and the intention is not to be limited to the details given herein. For example, the various elements or components may be combined or integrated in another system or certain features may be omitted, or not implemented. Method steps may be implemented in an order that differs from that presented.

Also, techniques, systems, subsystems and methods described and illustrated in the various implementations as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

While the above detailed description has shown, described, and pointed out the fundamental novel features of the disclosure as applied to various implementations, it will be understood that various omissions and substitutions and changes in the form and details of the system illustrated may be made by those skilled in the art, without departing from the intent of the disclosure.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow. In particular, materials and manufacturing techniques may be employed as within the level of those with skill in the relevant art. Furthermore, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

For the sake of clarity, the processes and methods herein have been illustrated with a specific flow, but it should be understood that other sequences may be possible and that some may be performed in parallel, without departing from the spirit of the invention.

All references cited herein are intended to be incorporated by reference. Although the present invention has been described above in terms of specific embodiments, it is anticipated that alterations and modifications to this invention will no doubt become apparent to those skilled in the art and may be practiced within the scope and equivalents of the appended claims. More than one computer may be used, such as by using multiple computers in a parallel or load-sharing attribute or distributing tasks across multiple computers such that, as a whole, they perform the functions of the components identified herein; i.e. they take the place of a single computer. Various functions described above may be performed by a single process or groups of processes, on a single computer or distributed over several computers. Processes may invoke other processes to handle certain tasks. A single storage device may be used, or several may be used to take the place of a single storage device. The present embodiments are to be considered as illustrative and not restrictive, and the invention is not to be limited to the details given herein. It is therefore intended that the disclosure and following claims be interpreted as covering all such alterations and modifications as fall within the true spirit and scope of the invention.

What is claimed is:

1. An apparatus for providing real-time multi-beam ultrasound imaging for guidance of a tool during a procedure comprising:
a transducer container including a plurality of transducers, and a tool-guiding channel for receiving an insertion tool for use during the procedure;
a housing comprising ultrasound beam processing and image processing circuits and a display, the transducer container rotatably coupled to the housing;
wherein the plurality of transducers are spaced laterally around the tool-guiding channel and are angled inboard in relation to the bottom surface of the transducer container, the bottom surface of the transducer container being adapted to allow transmission of ultrasound signals from the plurality of transducers and arranged at a fixed angle with respect to a longitudinal axis of the tool-guiding channel.

2. The apparatus of claim 1 wherein the ultrasound signals from the plurality of transducers are processed to form a single ultrasound image for displaying on the display.

3. The apparatus of claim 1 wherein the angle formed between each of the plurality of transducers and the bottom surface of the transducer container is within the range of five to fifty-five degrees.

4. The apparatus of claim 1 wherein the bottom surface of the transducer container is substantially perpendicular to the longitudinal axis of the tool-guiding channel.

5. The apparatus of claim 1 wherein the plurality of transducers are arranged to form an ultrasonic beam.

6. The apparatus of claim 1 wherein the angle between the plurality of transducers and the bottom surface of the transducer container allows the detection of liquid flow within a body of a patient.

7. The apparatus of claim 1 wherein the plurality of transducers are arranged to detect a position of the insertion tool that is inserted through the tool-guiding channel through skin of a patient.

8. The apparatus of claim 1 wherein the insertion tool is one of a needle or a cutting tool used for medical procedures.

9. The apparatus of claim 1 further comprising a body-attachment mechanism coupled to the housing for attaching the apparatus to a body of a patient during the procedure.

10. The apparatus of claim 9 wherein the body-attachment mechanism is one of a belt or a tape.

11. The apparatus of claim 1 further comprising a lockable attachment mechanism configured to allow locking of the rotatably attached transceiver container at a fixed position.

12. The apparatus of claim 1 wherein the tool-guiding channel includes an opening for receiving the insertion tool that has a diameter between 1 mm and 10 mm.

13. The apparatus of claim 1 further configured to be water proof.

14. The apparatus of claim 1 further configured to be resistant to shocks or vibrations.

15. The apparatus of claim 1 further comprising a gel layer extending at least partially over the outside surface of the transmitter container.

16. The apparatus of claim 1, wherein the transducer container is a disposable attachment.

17. The apparatus of claim 15, wherein the gel layer provides a cushion between the apparatus and a patient during the procedure.

18. The apparatus of claim 15 wherein the gel layer is made of a medical grade silicone.

19. The apparatus of claim 15, wherein the gel layer includes a cylindrical component that extends along an outside surface of the tool-guiding channel.

20. The apparatus of claim 15, wherein the gel layer includes at least one disposable component.

21. A method for detecting a needle position relative to tissues in a body comprising:
providing an ultrasound probe comprising a left and right transducer laterally spaced around a needle channel and angled inboard in relation to the probe; and
detecting via the left and right transducers the needle position within the tissues in the body, the needle inserted through the needle channel into the body; and
displaying a real-time ultrasound-generated image on a display attached to the probe providing the detected needle position.

22. The method of claim 21 wherein the left and right transducers are arranged to form one ultrasound-generated image.

23. The method of claim 21 wherein the angle formed between each of the left and right transducers and the bottom of the probe is within the range of five to fifty-five degrees.

24. The method of claim 21 wherein both the left and right transducers are arranged to form an ultrasonic beam.

25. The method of claim 21 wherein the angle of the transducers allows the detection of liquid flow within the body.
